(19) United States
(12) Patent Application Publication
(10) Pub. No.: US 2018/0119134 A1
(43) Pub. Date: May 3, 2018

(54) METHOD FOR SCREENING FOR PEPTIDE SEQUENCES THAT STIMULATE BACTERIAL GROWTH

(71) Applicant: BOARD OF REGENTS, THE UNIVERSITY OF TEXAS SYSTEM, Austin, TX (US)

(72) Inventor: Bryan DAVIES, Austin, TX (US)

(21) Appl. No.: 15/801,383

(22) Filed: Nov. 2, 2017

Related U.S. Application Data
(60) Provisional application No. 62/416,436, filed on Nov. 2, 2016.

(51) Int. Cl. C12N 15/10; C12Q 1/02

(52) U.S. Cl. CPC C12N 15/1037; C12Q 1/025

(57) ABSTRACT

Compositions and methods for isolating biologically active polypeptides (e.g., bacterial pro-growth polypeptides) are provided. In some aspects, bacterial cell populations are provided that express a surface-displayed library of candidate polypeptide sequences under the control of an inducible promoter.

This application claims the benefit of U.S. Provisional Patent Application No. 62/416,436, filed Nov. 2, 2016, the entirety of which is incorporated herein by reference.

### BACKGROUND OF THE INVENTION

### 1. Field of the Invention

The present invention relates generally to the field of molecular biology, microbiology and medicine. More particularly, it concerns transgenic bacteria and method for identifying polypeptides having a desired biological activity.

### 2. Description of Related Art

One the major unaddressed areas medicine involves the interaction between health and the populations of microbes that inhabit the human body. These microbes are now known to provide crucial benefits to the human host, such a providing synthesis of vitamins and helping to prevent infection with pathogenic microorganisms. However, despite their importance, to date there has been very limited ability to intervene in the growth or hemostasis of microbial populations living in an organism, such as a human. Thus, there remains a need for methods of identifying and characterizing new, effective, molecules that can effect the growth and health of microbial populations.

### SUMMARY OF THE INVENTION

In a first embodiment the invention provides a method for identifying a polypeptide having a desired biological activity comprising: obtaining a first population of bacterial cells, said cells comprising nucleic acid constructs encoding a fusion protein under the control of an inducible promoter, said fusion protein comprising a secretion signal sequence, a candidate polypeptide sequence, optionally, a linker sequence, and a bacterial membrane anchor sequence; inducing expression of the fusion protein in the bacterial cells; and identifying the candidate polypeptide sequences having the desired biological activity, wherein the biological activity is not an antibiotic activity. In some aspects, the desired biological activity comprises an activity that changes cell size, cell shape, cell adhesion (e.g., cell-cell adhesion or cell-surface adhesion), or provides a pro-growth or pro-survival activity. In further aspects, the desired biological activity comprises an activity that induce or repress gene activity (e.g., by directly affecting transcription factor activity or altering signal transduction pathways). In still further aspects, the desired biological activity may be a change in the growth conditions required by the bacteria. For example, a polypeptide having a desired biological activity may allow bacteria to tolerate low temperature conditions, high temperature conditions, low pH conditions, high pH conditions, low salt conditions, high salt conditions, low nutrient conditions, aerobic conditions, anaerobic conditions or conditions where there is an inhibitor or toxin (e.g., an antibiotic) present.

In some aspects, identifying the candidate polypeptide sequences comprises identifying the sequences from a first population of bacterial cells that are enriched in the population after incubation period. For example, the incubation period may involve exposure of the first population of bacterial cells to low temperature conditions, high temperature conditions, low pH conditions, high pH conditions, low salt conditions, high salt conditions, low nutrient conditions, aerobic conditions, anaerobic conditions or conditions where there is an inhibitor or toxin (e.g., an antibiotic) present. In still further aspects, a first population of bacterial cells are mixed with at least a second population of bacterial cells during the incubation period. For example, the first population of bacterial cells can be bacterial species found in normal gut microbial population of healthy humans and the second population of bacterial cells comprise pathogenic bacterial cells. In some aspects, an incubation period of the embodiments is at least 1 hour, 2, hours, 4 hours, 8 hours, 12 hours, 1 day, 2 days, 3 days, or one week (e.g., about 8 hours to one week).

In other aspects, identifying the candidate polypeptide sequences comprises separating cells based on the phenotype of the cells. For example, cells can be separated based on having a different shape or different adhesion properties. In further aspects, the method additionally comprises performing sequencing of the nucleic acid constructs in the population before said inducing step and performing sequencing from the intact cells after said inducing step to identify the candidate polypeptide sequences having a desired biological activity. In additional aspects, inducing expression of the fusion protein in the bacterial cells further comprises inoculating the bacterial cells into a test animal. For example the test animal can be a rat, mouse, guinea pig, pig, cow, horse, chicken or primate. In some aspects, inoculating bacterial population of the embodiments in inoculated into an animal by oral administration, intramuscular, intradermal, intraperitoneal, intracranial or intravenous injection or inoculation into the stomach or digestive tract.

In a further embodiment there is provided a recombinant bacterial cell, comprising a heterologous nucleic acid construct encoding a fusion protein under the control of an inducible promoter, said fusion protein comprising: (i) a secretion signal sequence; (ii) a candidate polypeptide sequence; (iii) optionally, a linker sequence; and (iv) a bacterial membrane anchor sequence. In related embodiment there is provided a population of bacterial cells, said cells comprising a heterologous nucleic acid construct encoding a fusion protein under the control of an inducible promoter, said fusion protein comprising a secretion signal sequence, a candidate polypeptide sequence, an optional linker sequence, and a bacterial membrane anchor sequence, wherein said population collectively comprise a plurality of different candidate polypeptide sequences. In some aspects, the majority of the bacterial cells of the population comprise nucleic acid constructs encoding 1, 2 or 3 different candidate polypeptide sequences. In still further aspects, the majority of the bacterial cells of the population comprise nucleic acid constructs encoding no more than 2 different candidate polypeptide sequences.

In certain aspects, an encoded fusion protein of the embodiments comprises, from N- to C-terminus: (i) a secretion signal sequence; (ii) a candidate polypeptide sequence; (iii) an optional linker sequence; and (iv) a bacterial membrane anchor sequence. In other aspects, the encoded fusion protein comprises, from N- to C-terminus: (i) a secretion signal sequence; (iv) a bacterial membrane anchor sequence; (iii) an optional linker sequence; and (ii) a candidate polypeptide sequence. In some aspects the population of bacterial cells comprises nucleic acid constructs encoding 1,000 to 100,000, 500,000, 1,000,000, 5,000,000 or 10,000,000 different candidate polypeptide sequences.

In still further aspects, obtaining the population of bacterial cells comprises transforming a population of bacterial cells with said nucleic acid constructs, wherein the nucleic acid constructs encode a plurality of different candidate polypeptide sequences. In particular aspects, the method further comprises mutating the identified sequences having a desired biological activity to generate nucleic acid constructs with mutated candidate polypeptide sequences and identifying mutated candidate polypeptide sequences having the desired biological activity in accordance with the embodiment described above.

The bacterial cells may be gram positive or gram negative bacterial cells. In certain aspects, of bacterial cells are a bacterial species found in normal tissue microbial population of a healthy human (e.g., a gut, mouth or skin population). In certain aspects the bacterial cells comprise bacteria from genus Acinetobacter, Bacteroides, Enterococcus, Escherichia, Enterobacter, Klebsiella, Bifidobacterium, Staphylococcus, Lactobacillus, Clostridium, Proteus, Pseudomonas, Salmonella, Faecalibacterium, Peptostreptococcus or Peptococcus. In some aspects the bacterial cells comprise Acinetobacter baumannii, Bacteroides thetaiotaomicron, Bacteroides fragilis, Bacteroides melaninogenicus, Bacteroides oxalis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enteritidis, Faecalibacterium prausnitzii, Peptostreptococcus sp. or Peptococcus sp. In some further aspects, the bacterial cells comprise, Bacillus anthraces, Bordetella pertussis, Borrelia burgdorferi, Brucella abortus, Brucella canis, Brucella melitensis, Brucella suis, Burkholderia cepacia, Burkholderia pseudomallei, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli (e.g., Enteropathogenic E. coli, Enterotoxigenic E. coli or E. coli O157:H7), Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitides, Pseudomonas aeruginosa, Rickettsia rickettsia, Salmonella typhi, Salmonella typhimurium, Shigella sonnei, Staphylococcus aureusa, Staphylococcus epidermidis, Staphylococcus saprophyticus, Streptococcus agalactiae, Streptococcus pneumonia, Streptococcus pyogenes, Treponema pallidum, Vibrio cholera, or Yersinia pestis cells. In some particular aspects, the bacterial cells are E. coli.

In still a further embodiment there is provided a recombinant DNA vector comprising a polypeptide coding sequence under the control of an inducible promoter, said polypeptide coding sequence comprising: (i) a sequence encoding a secretion signal sequence; (ii) a recombinant cloning site or a sequence encoding a candidate polypeptide; (iii) a sequence encoding a linker sequence; and (iv) a sequence encoding a bacterial membrane anchor sequence. In some aspects, the recombinant cloning site comprises a restriction endonuclease recognition sequence or a recombinase recognition site (e.g., a Cre recombinase recognition site). For example, in some cases, the recombinant cloning site comprises a multiple cloning site comprising a plurality of restriction endonuclease recognition sequences (e.g., 2, 3, 4, 5 or more different endonuclease recognition sequences). In preferred aspects, recombinant cloning site of the embodiments is arranged such that, after insertion of a candidate polypeptide coding sequence into the site, a fusion protein comprising sequence encoding the secretion signal, the candidate polypeptide and the membrane anchor is produced.

In a related embodiment there is provided a library of DNA vectors, each member of the library comprising a polypeptide coding sequence under the control of an inducible promoter, said polypeptide coding sequence comprising: (i) a sequence encoding a secretion signal sequence; (ii) a sequence encoding a candidate polypeptide; (iii) a sequence encoding a linker sequence; and (iv) a sequence encoding a bacterial membrane anchor sequence. For example, in some aspects, a library of the embodiments comprises DNA vectors encoding 1,000 to 100,000, 500,000, 1,000,000, 5,000,000 or 10,000,000 different candidate polypeptide sequences.

Certain aspects of the embodiments concern fusion proteins comprising a secretion signal sequence, a candidate polypeptide sequence, an optional linker sequence, and a bacterial membrane anchor sequence. In particular aspects, the bacterial membrane anchor sequence can be a portion of a lipoprotein, an outer membrane protein or a component of the cell surface. In some aspects, the membrane anchor sequence is from a gram positive or gram negative bacteria. In certain aspects, the candidate polypeptide sequence may be from about 10, 15, 20, 25, 30, 35, 40, 45, 50, 100, 150 to 200 amino acids in length. In further aspects, the bacterial membrane anchor sequence comprises the membrane anchor sequence from OmpA. In some aspects, the bacterial membrane anchor sequence comprises a sequence at least 90%, 91%, 92%, 93%, 94%, 95%, 96%, 97%, 98%, 99% or 100% identical to SEQ ID NO: 1 (NPYVGFEMGYDWLGRMPYKGSVENGAYKAQGVQLTAKLGYPITDDLDIYTRLGG MVWRADTKSNVYGKNHDTGVSPVFAGGVEYAITPEIATRLEYQWTNNIGDAHTIGT RPDN).

In still further aspects, the secretion signal of a fusion protein is from a gram positive or gram negative bacteria. In some specific aspects, the signal sequence is from murein lipoprotein (Lpp). In certain aspects, the secretion signal sequence comprises a sequence at least 90%, 91%, 92%, 93%, 94%, 95%, 96%, 97%, 98%, 99% or 100% identical to SEQ ID NO: 2 (MKATKLVLGAVILGSTLLAGCSSNAKIDQ).

In yet still further aspects, a fusion protein of the embodiments comprises an optional linker sequence. In some aspects, the linker sequence may comprise two or more Gly positions or a poly Gly sequence. In certain aspects, a linker sequence comprises at least 5, 10, 15, 20, 25, 30, 35 or 40 amino acids. For example, the linker sequence can be from about 10 to 100 amino acids in length. In particular aspects, the linker sequence comprises a sequence at least 90%, 91%, 92%, 93%, 94%, 95%, 96%, 97%, 98%, 99% or 100% identical to SEQ ID NO: 3 (SQEPAAPAAEATPAAEAPASEAPAAEAAPADAAEAPAAGI). In other aspects, the linker sequence comprises at least two repeats of a sequence at 90% identical to SEQ ID NO: 3.

In still further aspects, a nucleic acid construct of the embodiments further comprises a transcription terminator after the sequence encoding the fusion protein. In certain aspects, the transcription terminator is the rrnB terminator. In yet still further aspects, the nucleic acid construct additionally comprises a selectable marker. The selectable marker may be a drug resistance marker.

In further aspects, the inducible promoter is a drug inducible promoter. In particular aspects, inducing expression of the fusion protein comprises applying a drug to the population, said drug inducing the promoter. In specific aspects, the drug inducible promoter is an Isopropyl β-D-1-thiogalactopyranoside (IPTG) inducible promoter.

In still yet further aspects, the inducible promoter is a promoter that is induced by environmental conditions, such as pH, salt, temperature, or anoxic conditions. In certain aspects, the inducible promoter is activated at a site of infection. In other aspects, the inducible promoter is a promoter from a bacterial virulence gene. In a specific aspect, the promoter may be V. cholerae virulence promoter. In certain aspects, the nucleic acid construct further comprises a selectable marker.

In still yet a further embodiment, the invention provides a laboratory animal comprising a bacterial cell or population of bacterial cells as described by the embodiments and aspects herein.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic shows an example antimicrobial peptide surface display system comprised of (1) Lpp signal sequence, (2) OmpA transmembrane helices (membrane anchor), (3) flexible tether (linker), and (4) a C-terminal peptide (candidate polypeptide). Induction of the system leads to surface display of the C-terminal antimicrobial peptide and cell lysis, which eliminates the expressing bacterium from the population.

FIGS. 2A-2B: In vivo peptide screening. (A) Expression of the peptide surface display system is placed under control of a V. cholerae virulence promoter. (B) White V. cholerae expresses a non-lethal control peptide. Blue V. cholerae expresses a lethal AMP. Neither peptide is expressed in vitro. The input inoculum contains both white and blue V. cholerae at a 1:1 ratio. This input mixture is inoculated in a mouse. In the mouse, the virulence promoter is activated and drives expression of the peptide surface display system. There is no effect on white V. cholerae. Blue V. cholerae expressing the lethal AMP is killed inside the mouse resulting in greatly diminished presence in the output bacteria quantitation.

FIGS. 3A-3B: Pro-growth peptide identification. In the library screening, peptides that become depleted from the pool have potential antimicrobial activity (lower right triangle). Conversely, peptides that become enriched in the pool have potential pro-growth activity (upper left triangle).

FIG. 4: Activity of pro-growth peptide. A 1:1 culture of both bacteria were treated with a control or a putative pro-growth peptide for 1 h and then plated to count colony forming units. The pro-growth peptide increased the number of E. coli CFU recovered.

### DESCRIPTION OF ILLUSTRATIVE EMBODIMENTS

### I. The Present Embodiments

The methods provided herein address the need for libraries that may be used to screen for polypeptide having desired biological activities. For example, the inventors have developed a high-through system for identifying and selecting polypeptide sequences that promote bacterial cell growth and survival. Importantly, the system is able to identify polypeptides that operate on the exterior of the cell and do not, therefore, require additional modifications (i.e., to allow the polypeptides to enter into cells). However, despite the exterior display of library sequences, the system is surprisingly able to identify sequences having very specific biological activities, such a growth enhancing properties. This new screening methodology can be adapted for use in a wide range of gram positive and gram negative bacterial systems and therefore can be used to identify new biologically active polypeptides specific for any bacteria of interest. Moreover, the methods detailed herein allow polypeptides to be selected from highly diverse libraries, which allows for large numbers of candidate sequences to be efficiently identified and characterized in a very short time span. Moreover, the ability of the system to function in vivo allows for the identification of sequences that have relevance in actual animal models, rather than merely in a contrived in vitro environment. Thus, identified polypeptides are much more likely lead to relevant therapeutic molecules.

### II. Nucleic Acid-Based Expression Systems

A wide range of nucleic acid-based expression systems may be used for the expression of candidate polypeptides. For example, one embodiment of the invention involves transformation of bacteria with the coding sequences of fusion polypeptides comprising a candidate polypeptide linked to a membrane anchor sequence and section signal. Numerous expression systems exist that comprise some or all of the sequence components discussed below.

Vectors may find use with the embodiments, for example, in the transformation of bacterial cells with a nucleic acid sequences encoding a candidate polypeptide which one wishes to screen for a desired biological activity. In one embodiment of the invention, an entire heterogeneous “library” of nucleic acid sequences encoding candidate polypeptides may be introduced into a population of bacteria, thereby allowing screening of the entire library. The term “vector” is used to refer to a carrier nucleic acid molecule into which a nucleic acid sequence can be inserted for introduction into a cell where it can be replicated. A nucleic acid sequence can be “exogenous,” or “heterologous”, which means that it is foreign to the cell into which the vector is being introduced or that the sequence is homologous to a sequence in the cell but in a position within the host cell nucleic acid in which the sequence is ordinarily not found. Vectors include plasmids, bacteriophages, and artificial chromosomes. However, in preferred aspects, vectors for use according to the embodiments are plasmid vectors, which do not integrate in the genome of host bacterial cells. An examples of such an expression system is the pET Expression System and an E. coli expression system. A plasmid-based inducible expression system for use in gram positive bacteria, such as Staphylococcus aureus, is likewise detailed in Liew et al., 2011, which is incorporated herein by reference. One of skill in the art may construct a vector through standard recombinant techniques, which are described in Maniatis et al., 1988 and Ausubel et al., 1994, both of which are incorporated herein by reference.

The term “expression vector” refers to a vector containing a nucleic acid sequence coding for at least part of a gene product capable of being transcribed and then translated into a polypeptide. Expression vectors can contain a variety of “control sequences,” which refer to nucleic acid sequences necessary for the transcription and possibly translation of an operably linked coding sequence in a particular host organism (e.g., gram positive or gram negative bacteria). In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Preferably a promoter a promoter for use according to the embodiments is a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. Furthermore, it is contemplated that the control sequences that direct transcription and/or expression of sequences within non-nuclear organelles such as mitochondria, chloroplasts, and the like, can be employed as well.

In preferred aspects, a promoter (or promoter enhancer system) for use according to the embodiments is an inducible promoter that provides expression of a sequence based on an external stimulus. For example, the inducible promoter may be a promoter that provides expression only in the presence of a particular compound (e.g., IPTG), at a particular pH, or in specific environmental (e.g., lighting) conditions.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Termination Signals

The vectors or constructs prepared in accordance with the present invention will generally comprise at least one termination signal. A “termination signal” or “terminator” is comprised of the DNA sequences involved in specific termination of an RNA transcript by an RNA polymerase. Thus, in certain embodiments, a termination signal that ends the production of an RNA transcript is contemplated. A terminator may be necessary in vivo to achieve desirable message levels.

Terminators contemplated for use in the invention include any known terminator of transcription described herein or known to one of ordinary skill in the art, including but not limited to, for example, rhp dependent or rho independent terminators. In certain embodiments, the termination signal may be a lack of transcribable or translatable sequence, such as due to a sequence truncation.

5. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

6. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker, such an antibiotic resistance marker.

7. Fusion Polypeptides

As described above, in some aspects a vector of the embodiments comprises a sequence for expression, which comprises a fusion of a membrane anchor sequence and a candidate polypeptide sequence. Furthermore, in some aspects, the fusion polypeptide comprises a secretion signal that directs the fusion protein to the bacterial (outer) membrane. Optionally, the fusion polypeptide further comprises a linker positions between the candidate polypeptide sequence and the membrane anchor sequence.

a. Signal Sequences

In some aspects, a fusion polypeptide of the embodiments comprises a signal sequence that targets the fusion polypeptide to the membrane (and may be cleaved away from the fusion). In certain aspects, the secretion signal sequence is from a gram positive bacteria. In other aspects, the signal sequence can be from a gram negative bacteria (e.g., E. coli). For example, the signal sequence can be from murein lipoprotein (Lpp). In certain aspects, the secretion signal sequence comprises a sequence at least 90%, 91%, 92%, 93%, 94%, 95%, 96%, 97%, 98%, 99% or 100% identical to SEQ ID NO: 2. Further aspects, the signal sequence can be a signal sequence from an autotransporter polypeptide of a gram negative bacteria. For example, the signal sequence can be from AIDA-I, EstA, MisL, Hbp, Ag43, BrkA, OmpA, OmpC, OmpX, LamB, FhuA, Pfal, EspP, IgAP, Pet or Yfal (see, e.g., Nicolay et al., 2015 and van Bloois et al., 2011, each incorporated herein by reference).

b. Membrane Anchor Sequence

Certain aspect of the embodiments concern fusion polypeptides that comprise a bacterial membrane anchor sequence. For example, the membrane anchor sequence can be composed of all or part of an integral membrane protein from a gram negative or gram positive bacteria. In further aspects, the membrane anchor sequence can be a non-integral membrane polypeptide, such as a lipoprotein or a component of a bacterial surface appendage, caspule or cell wall. In particular aspects, the bacterial membrane anchor sequence can be an outer membrane anchor sequence. In some aspects, the sequence can be a beta-barrel domain from an autotransporter polypeptide of a gram negative bacteria. For example, the membrane anchor sequence can comprise a membrane anchor domain from AIDA-I, EstA, MisL, Hbp, Ag43, BrkA, OmpA, OmpC, OmpX, LamB, FhuA, Pfal, EspP, IgAP, Pet, Yfal or MraY (see, e.g., Nicolay et al., 2015 and van Bloois et al., 2011, each incorporated herein by reference). In further aspects, the bacterial membrane anchor sequence comprises the membrane anchor sequence from OmpA. In some aspects, the bacterial membrane anchor sequence comprises a sequence at least 90%, 91%, 92%, 93%, 94%, 95%, 96%, 97%, 98%, 99% or 100% identical to SEQ ID NO: 1.

c. Linker Sequence

It will be understood that in certain cases, a fusion polypeptide may comprise additional amino acids positioned between the candidate polypeptide sequence and the membrane anchor sequence. In general these sequences are interchangeably termed “linker sequences” or “linker regions.” One of skill in the art will recognize that linker regions may be one or more amino acids in length and often comprise one or more glycine residue(s) which confer flexibility to the linker. A variety of linkers can be used as part of fusion polypeptide of the embodiments. In preferred aspects, the optional linker sequence is positioned between the membrane anchor sequence and the candidate polypeptide sequence. In certain aspects the linker sequence comprises at least or about 1, 2, 3, 4, 5, 6, 7, 8, 9, 10, 11, 12, 13, 14, 15, 16, 17, 18, 19 or 20 amino acids. In still further aspects the linker comprises between about 10 and 200, 10 and 100, 20 and 100, 40 and 100 or 50 and 90 amino acids.

In certain aspects, the linker sequence may comprise two, three, four or more Gly positions or a poly Gly sequence having two or more consecutive Gly positions. In particular aspects, the linker sequence comprises a sequence at least 90%, 91%, 92%, 93%, 94%, 95%, 96%, 97%, 98%, 99% or 100% identical to SEQ ID NO: 3; SEQ ID NO: 4 (GSTSGSGKPGSGEGSTKG); SEQ ID NO: 5 (EAAAK); or SEQ ID NO: 6 (GGGGS). In still further aspects, a linker comprises two, three or more repeats of a sequence at least 90%, 91%, 92%, 93%, 94%, 95%, 96%, 97%, 98%, 99% or 100% identical to SEQ ID NO: 3; SEQ ID NO: 4; SEQ ID NO: 5; or SEQ ID NO: 6. In some cases, such linker sequences can be repeated 1, 2, 3, 4, 5, 6, or more times or combined with one or more different linkers to form an array of linker sequences. For example, the linker sequence can comprise two consecutive repeats of a sequence according to SEQ ID NO: 4.

In still further aspects, the linker sequence can comprise all or part of a bacterial membrane polypeptide (e.g., a gram negative outer membrane polypeptide). In some aspects, the linker is a portion of sequence from a Neisseria polypeptide. For example, the linker can comprise 10, 15, 20, 25, 30, 35, 40 or more consecutive amino acid from any one of SEQ ID NOs:7-26. In still further aspects, the linker comprises a sequence at least 90%, 91%, 92%, 93%, 94%, 95%, 96%, 97%, 98%, 99% or 100% identical to any one of SEQ ID NOs:7-26.

In further aspects, a linker sequence may comprise a protease cleavage site, such as the cleavage site recognized by an extracellular protease. In still further aspects, a protease cleavage site can be a site that is by a recombinant protease. In certain aspects, a linker can comprise cleavage site that is cleaved by urokinase, plasmin, thrombin, Factor IXa, Factor Xa, or a metalloproteinase, such as collagenase, gelatinase, or stromelysin.

d. Candidate Biologically Active Polypeptide

In certain aspects, the candidate polypeptide sequence(s) may be from about 10, 15, 20, 25, 30, 35, 40, 45, 50, 60, 70, 80, 90, 100, 125, 150, 175, 200 to about 300 amino acids in length. In some aspects, the candidate polypeptide sequences can be a sequence based on a known polypeptide (e.g., a polypeptide having a known pro-growth activity) that has been randomly or selectively mutated. In further aspects, candidate polypeptide sequences can be a randomized group of sequences.

### III. Host Cells

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

In particular aspects, a host cell is a Gram negative bacterial cell. In still further aspects, the host cell is a gram positive bacterial cell. For example, in some aspects, the host cell can a human bacterial pathogen such as a Acinetobacter baumannii, Bacillus anthraces, Bacteroides thetaiotaomicron Bacteroides fragilis, Bacteroides melaninogenicus, Bacteroides oxalis, Bordetella pertussis, Borrelia burgdorferi, Brucella abortus, Brucella canis, Brucella melitensis, Brucella suis, Burkholderia cepacia, Burkholderia pseudomallei, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli (e.g., Enteropathogenic E. coli, Enterotoxigenic E. coli or E. coli O157:H7), Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitides, Pseudomonas aeruginosa, Rickettsia rickettsia, Salmonella typhi, Salmonella typhimurium, Shigella sonnei, Staphylococcus aureusa, Staphylococcus epidermidis, Staphylococcus saprophyticus, Streptococcus agalactiae, Streptococcus pneumonia, Streptococcus pyogenes, Treponema pallidum, Vibrio cholera, or Yersinia pestis bacterial cell.

Numerous prokaryotic cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for bacteriophage.

Many host cells from various cell types and organisms are available and would be known to one of skill in the art. Similarly, a viral vector may be used in conjunction with a prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Peptide-Screening Platform

The present platform creates microenvironments for individual bacteria and peptide sequences to interact under physiologically relevant conditions, within a bacterial population, such as a mixed population. Effects of the candidate polypeptides of cell physiology (e.g., growth properties, cell-cell or cell-surface adhesion, cell shape or membrane transport, or gene regulation) can be measured using next-generation sequencing, allowing rapid and batch screening of millions of peptides for antimicrobial activity in a single tube.

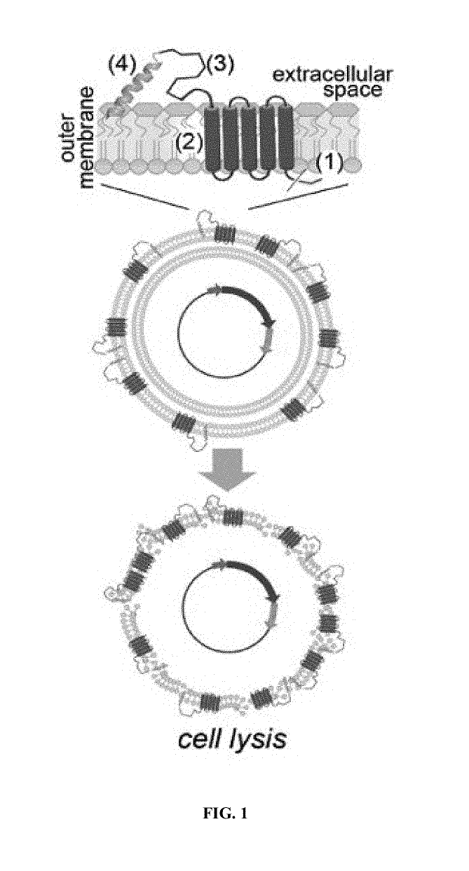

A peptide-screening platform (exemplified in FIG. 1) is engineered from four primary components: (1) the murein lipoprotein (Lpp) signal sequence that directs proteins for export from the cytoplasm and is subsequently cleaved, (2) five loops of the gram-negative outer membrane protein OmpA that anchors our system to the cell surface, (3) a flexible tether (optional) that allows spatial freedom past the outer membrane, and (4) a C-terminal peptide. The bacterial transport machinery utilized by this system ensures that the construct does not interact with cellular components until it is localized to the surface, alleviating the burden of intracellular toxicity. The unstructured, low-complexity-region protein was engineered from the Neisseria genus into this system to act as a flexible tether connecting the membrane anchoring OmpA component to the C-terminal peptide. Two low-complexity-region proteins fused together (2× tether) produces a flexible tether that extends up to 20 nm. This length allows C-terminal peptides to interact with the cell surface and also to penetrate the cell surface and interact with periplasmic components. Through these interactions, C-terminal peptides exhibiting antimicrobial activity lyse the bacteria expressing them thereby eliminating themselves from the population.

### Example 2—Screening Experiments Using the Platform

The screening system of the present disclosure is biologically encoded such that the screening can conducted in vivo at the site of the infection as well as in vitro. In general, the method comprises identifying the bacterium for which biologically active peptides are to be developed, identifying a promoter in the target bacterium that is activated in vivo when the bacterium is inside the host organism, exchanging the IPTG inducible promoter for the in vivo activated promoter, constructing the desired peptide library so that each peptide is only expressed inside the host, and infecting the host to allow infection to occur. Finally, the remaining peptides are recovered and sequenced to identify the peptides that have antimicrobial activity in vivo.

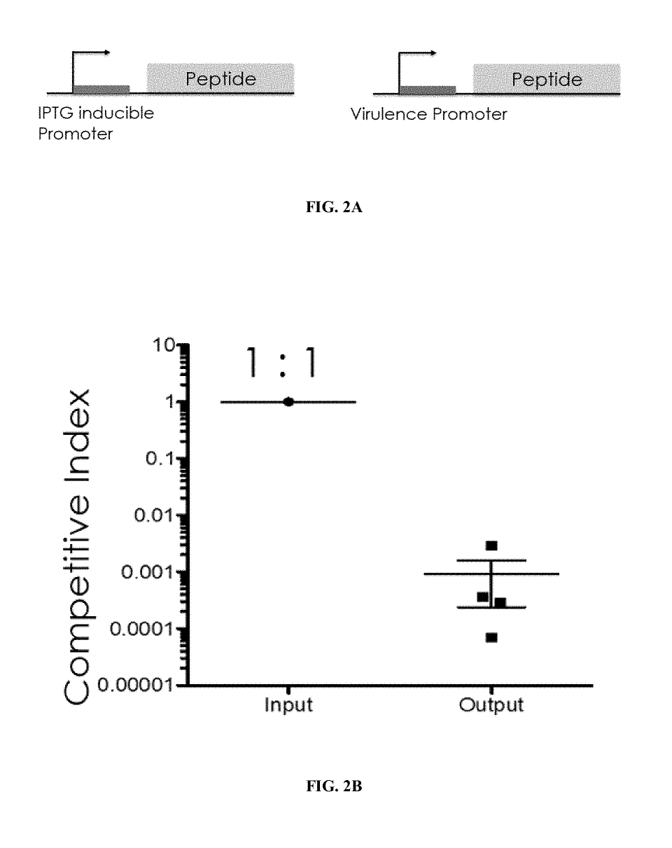

For a proof of concept experiment, the pathogen Vibrio cholera, that causes intestinal infection, was used. The tcpA promoter is activated when V. cholerae is in the intestine. A control (HA) or antimicrobial peptide were cloned into the surface display construct, under the expression of the tcpA promoter. The V. cholerae expressing the control peptide was lacZ− (white). The V. cholerae expressing the antimicrobial peptide was lacZ+ (blue). When mixed in a 1:1 ratio, both strains grow equally well in liquid culture, since the tcpA promoter is not active and the peptides are not expressed (FIGS. 2A-2B). The 1:1 mixture is inoculated into a mouse intestine and allowed to grow for 18 h. The bacteria are then recovered and innumerated. In the intestine, the tcpA promoter activates expression of the peptide surface display system. The blue V. cholerae expressing the antimicrobial peptide eliminate themselves and are not recovered from the intestine.

The isogenic E. coli strain WD101 carries a mutation in its lipopolysaccharide layer in the outer membrane that makes it resistant to antimicrobial peptides′. The platform used a plasmid with a broad host range origin of replication offering flexibility to identify peptides in many gram-negative bacteria. For example, A. baumannii is a gram-negative, hospital-associated pathogen that quickly acquires antibiotic resistance. Without any modifications, the methods described herein can move the screened—V. cholerae surface display construct into A. baumannii and show that it functions in a similar manor to E. coli. The selective marker on this plasmid can be changed to allow its use in bacteria with different functions in vivo.

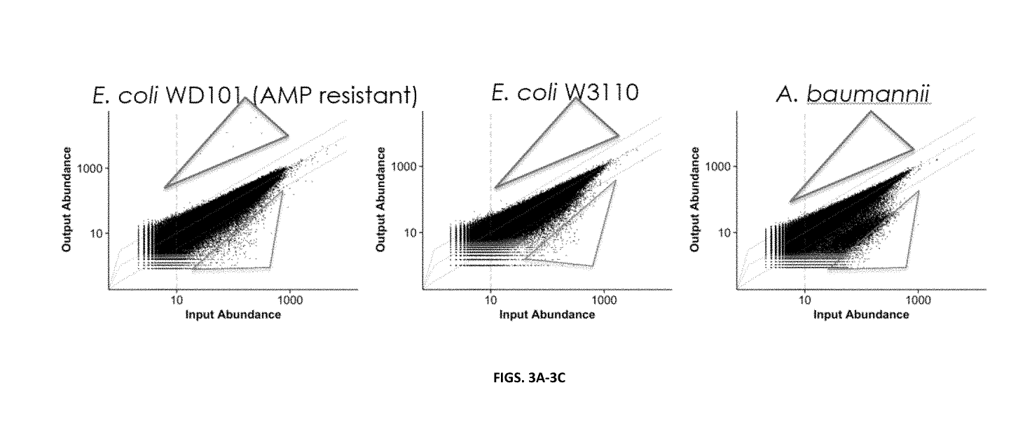

A small ˜90,000-clone peptide library was generated by cloning a random 60 nucleotide peptide coding DNA duplex into the surface expression system of the embodiments. The library was transformed into the antimicrobial peptide resistant E. coli strain WD101. Then plasmids were isolated from intact bacteria. Sequencing reads for input and output samples were acquired, and the output samples were computationally translated and compared to the input library to identify peptides that were depleted after the incubation. FIGS. 3A-3B show the abundance of each peptide in the input library plotted against its abundance in a representative output library. The peptides that were depleted from the pool had potential antimicrobial activity while the peptides that were enriched in the pool had potential pro-growth activity.

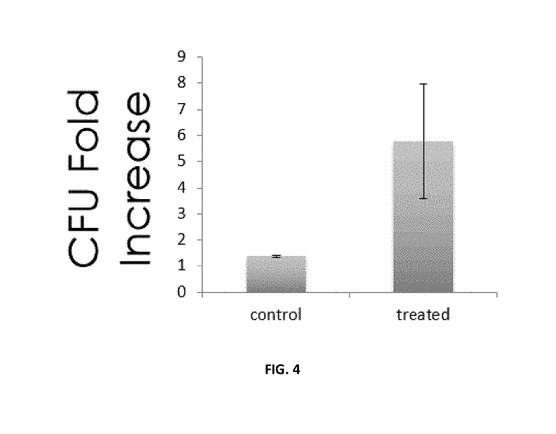

The pro-growth Peptide A was chemically synthesized and its activity was tested against E. coli in vitro. E. coli was incubated with the putative pro-growth Peptide A for 1 hour, then serially diluted in 10-fold increments and plated to determine the remaining number of viable bacteria. FIG. 4 demonstrates that the Pro-growth Peptide A effectively increases the number of E. coli CFU recovered.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ausubel et al., In: Current Protocols in Molecular Biology, John, Wiley & Sons, Inc, N Y, 1994.
Bahar, A. A. & Ren, D., Antimicrobial Peptides; Pharmaceuticals 6, 1543-1575, 2013.
Bergen, P. J. et al., ‘Old’ antibiotics for emerging multidrug-resistant bacteria; [Miscellaneous Article]. Curr. Opin. Infect. Dis. December 2012 25, 626-633, 2012.
Carbonelli et al., FEMS Microbiol Lett., 177:75-82. 1999.
CDC. Antibiotic Resistance Threats in the United States. (U.S. Department of Health and Human Services, 2013). at <http://www.cdc.gov/drugresistance/threat-report, 2013.
Cherkasov, A. et al., Use of Artificial Intelligence in the Design of Small Peptide Antibiotics Effective against a Broad Spectrum of Highly Antibiotic-Resistant Superbugs. ACS; Chem. Biol. 4, 65-74, 2009.
Clatworthy, A. E. et al., Targeting virulence: a new paradigm for antimicrobial therapy; Nat. Chem. Biol. 3, 541-548, 2007.
Cocea, Biotechniques, 23(5):814-816, 1997.
Deslouches, B. et al., De Novo Generation of Cationic Antimicrobial Peptides: Influence of Length and Tryptophan Substitution on Antimicrobial Activity; Antimicrob. Agents Chemother. 49, 316-322, 2005.
Fagerlund, A., Myrset, A. H. & Kulseth, M. A. in Therapeutic Peptides (ed. Nixon, A. E.) 19-33 (Humana Press). at http://link.springer.com/protocol/10.1007/978-1-62703-673-3 2, 2014.
Fischbach, M. A. & Walsh, C. T, Antibiotics for emerging pathogens; Science 325, 1089-1093, 2009.
Fjell, C. D., Designing antimicrobial peptides: form follows function; Nat. Rev. Drug Discov. 11, 37-51, 2012.
Fox, J. L., et al., Antimicrobial peptides stage a comeback; Nat. Biotechnol. 31, 379-382, 2013.
Fu, J. et al., Full-length RecE enhances linear-linear homologous recombination and facilitates direct cloning for bioprospecting; Nat. Biotechnol. 30, 440-446, 2012.
Gould, I. M. & Bal, A. M., New antibiotic agents in the pipeline and how they can help overcome microbial resistance; Virulence 4, 185-191, 2013.
Guilhelmelli, F. et al., Antibiotic development challenges: the various mechanisms of action of antimicrobial peptides and of bacterial resistance; Antimicrob. Resist. Chemother. 4, 353, 2013.
Guralp, S. A. et al., From Design to Screening: A New Antimicrobial Peptide Discovery Pipeline; PLoS ONE 8, e59305, 2013.
Hartigan, J., Compound Profiling: size impact on primary screening libraries. Spring 10; Drug Discovery World, http://www.ddw-online.com/p-92823, 2010.
Hilpert, K., et al., Peptide arrays on cellulose support: SPOT synthesis, a time and cost efficient method for synthesis of large numbers of peptides in a parallel and addressable fashion; Nat. Protoc. 2, 1333-1349, 2007.
Klevens, R. M. et al. Estimating health care-associated infections and deaths in U.S. hospitals, 2002. Public Health Rep. Washington DC 1974 122, 160-166, 2007.
Lee, J. Y. et al., Antibacterial peptides from pig intestine: isolation of a mammalian cecropin; Proc. Natl. Acad. Sci. 86, 9159-9162, 1989.
Levenson et al., 1998.
Liew et al., Microbiol., 157:666-676, 2011.
Liu, Z. et al., Length Effects in Antimicrobial Peptides of the (RW)n Series; Antimicrob. Agents Chemother. 51, 597-603, 2007.
Maniatis, et al., Molecular Cloning, A Laboratory Manual, Cold Spring Harbor Press, Cold Spring Harbor, N.Y., 1988.
Man, A. K. et al., Antibacterial peptides for therapeutic use: obstacles and realistic outlook; Curr. Opin. Pharmacol. 6, 468-472, 2006.
Nicolay et al., Crit. Rev. Microbiol., 41(1):109-123, 2015
O'Neil, J. Antimicrobial Resistance: Tackling a crisis for the health and wealth of nations. (United Kingdom, 2014).
Otvos, L., Peptide-Based Drug Design (ed. Otvos, L.) 1-8 (Humana Press) at http://link.springer.com/protocol/10.1007/978-1-59745-419-3_1, 2008.
Peschel, A. & Sahl, H.-G, The co-evolution of host cationic antimicrobial peptides and microbial resistance; Nat. Rev. Microbiol. 4, 529-536, 2006.
Rathinakumar, R. & Wimley, W. C., High-throughput discovery of broad-spectrum peptide antibiotics; FASEB J. 24, 3232-3238, 2010.
Sivertsen, A. et al., Synthetic cationic antimicrobial peptides bind with their hydrophobic parts to drug site II of human serum albumin; BMC Struct. Biol. 14, 4, 2014.
Upton, M., Cotter, P. & Tagg, J., Antimicrobial Peptides as Therapeutic Agents; Int. J. Microbiol. 2012, e326503, 2012.
Van Bloois et al., Applied Microbiol. 29(2):79-86, 2011.
Wiesner, J. & Vilcinskas, A., Antimicrobial peptides: The ancient arm of the human immune system; Virulence 1, 440-464, 2010.

1. A method for identifying a polypeptide having a desired biological activity comprising:
(a) obtaining a first population of bacterial cells, said cells comprising a nucleic acid construct encoding a fusion protein under the control of an inducible promoter, said fusion protein comprising:
(i) a secretion signal sequence;
(ii) a candidate biologically active polypeptide sequence;
(iii) a linker sequence; and
(iv) a bacterial membrane anchor sequence;
(b) inducing expression of the fusion protein in the bacterial cells; and
(c) identifying the candidate polypeptide sequences having a biological activity of interest, wherein the biological activity of interest is an not an antibiotic activity.

2. The method of claim 1, wherein the first population of bacterial cells are a bacterial species found in normal gut microbial population of healthy humans.

3. The method of claim 1, wherein the biological activity of interest is a pro-growth or pro-survival activity.

4. The method of claim 3, wherein identifying the candidate polypeptide sequences comprises identifying the sequences from bacterial cells that are enriched in the first population after an incubation period.

5. The method of claim 4, wherein the incubation period comprises exposure of the first population of bacterial cells to low temperature conditions, high temperature conditions, low pH conditions, high pH conditions, low salt conditions, high salt conditions, low nutrient conditions, high nutrient conditions, aerobic conditions, anaerobic conditions or conditions where there is an inhibitor or toxin present.

6. (canceled)

7. The method of claim 4, wherein the first population of bacterial cells are mixed with at least a second population of bacterial cells during the incubation period.

8-11. (canceled)

12. The method of claim 4, further comprising performing sequencing of the nucleic acid constructs in the first population before said inducing step, and performing sequencing on cells from the first population cells growth period to identify the candidate polypeptide sequences having a pro-growth or pro-survival activity.

13. The method of claim 1, wherein the encoded fusion protein comprises, from N- to C-terminus: (i) a secretion signal sequence; (ii) a candidate biologically active polypeptide sequence; (iii) a linker sequence; and (iv) a bacterial membrane anchor sequence.

14. The method of claim 1, wherein the encoded fusion protein comprises, from N- to C-terminus: (i) a secretion signal sequence; (iv) a bacterial membrane anchor sequence; (iii) a linker sequence; and (ii) a candidate biologically active polypeptide sequence.

15. The method of claim 1, wherein the first population of bacterial cells comprises nucleic acid constructs encoding 1,000 to 10,000,000 different candidate biologically active polypeptide sequences.

16. (canceled)

17. The method of claim 1, wherein obtaining the first population of bacterial cells comprises transforming a population of bacterial cells with said nucleic acid constructs, wherein the nucleic acid constructs encode a plurality of different candidate biologically active polypeptide sequences.

18. The method of claim 1, wherein the majority of the bacterial cells of the population comprise nucleic acid constructs encoding 2 different candidate biologically active polypeptide sequences.

19. The method of claim 1, further comprising mutating the identified sequences having the biological activity of interest to generate nucleic acid constructs with mutated candidate polypeptide sequences and identifying mutated candidate biologically active polypeptide sequences having the biological activity of interest in accordance with claim 1.

20-25. (canceled)

26. The method of claim 1, wherein the bacterial membrane anchor sequence comprises the membrane anchor sequence from OmpA.

27. The method of claim 1, wherein the bacterial membrane anchor sequence comprises a sequence at least 90% identical to SEQ ID NO: 1 (NPYVGFEMGYDWLGRMPYKGSVENGAYKAQGVQLTAKLGYPITDDLDIYTRLGGMV WRADTKSNVYGKNHDTGVSPVFAGGVEYAITPEIATRLEYQWTNNIGDAHTIGTRPDN).

28. The method of claim 1, wherein the secretion signal sequence is from murein lipoprotein (Lpp).

29. The method of claim 1, wherein the secretion signal sequence comprise a sequence at least 90% identical to SEQ ID NO: 2 (MKATKLVLGAVILGSTLLAGCSSNAKIDQ).

30. The method of claim 1, wherein the linker sequence comprises two or more Gly positions.

31. (canceled)

32. The method of claim 1, wherein the linker comprises a protease cleavage site.

33. The method of claim 1, wherein the linker sequence comprises a sequence at least 90% identical to SEQ ID NO: 3 (SQEPAAPAAEATPAAEAPASEAPAAEAAPADAAEAPAAGI).

34. (canceled)

35. The method of claim 1, wherein the nucleic acid construct further comprises a transcription terminator after the sequence encoding the fusion protein.

36-45. (canceled)


SEQUENCE LISTING (26 sequences)

SEQ ID NO: 1 - protein, 114 aa, Artificial sequence
  Synthetic polypeptide
NPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 2 - protein, 29 aa, Artificial sequence
  Synthetic polypeptide
MKATKLVLGAVILGSTLLAGCSSNAKIDQ

SEQ ID NO: 3 - protein, 40 aa, Artificial sequence
  Synthetic polypeptide
SQEPAAPAAEATPAAEAPASEAPAAEAAPADAAEAPAAGI

SEQ ID NO: 4 - protein, 18 aa, Artificial sequence
  Synthetic peptide
GSTSGSGKPGSGEGSTKG

SEQ ID NO: 5 - protein, 5 aa, Artificial sequence
  Synthetic peptide
EAAAK

SEQ ID NO: 6 - protein, 5 aa, Artificial sequence
  Synthetic peptide
GGGGS

SEQ ID NO: 7 - protein, 201 aa, Neisseria meningitidis
MHFDFCKTEYYFIDWRFVMKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEEASLTLDPAKLADGEYKFACTFPGHGALMNGKVTLVD

SEQ ID NO: 8 - protein, 201 aa, Neisseria meningitidis
MHFDFCKTEYYFIDWRFTMKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 9 - protein, 183 aa, Neisseria polysaccharea
MKTYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKITLVD

SEQ ID NO: 10 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKTSMGHNIVIGKTEDMDGIFKDGVGAAGTDYVKPDDARVVAHTKLIGGGEESSLTLDPAKLADGDYKFACTFPGHGALMNGKITLVD

SEQ ID NO: 11 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGIQPKTSMGHNIVIGKTEDMDGIFKDGVGAADTDYVKPDDARVVAHTKLIGGGEESSLTLDPAKLADGEYKFACTFPGHGALMNGKVTLVD

SEQ ID NO: 12 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKTSMGHNIVIGKTEDMDGIFKDGVGAADTDYVKPDDARVVAHTKLIGGGEESSLTLDPAKLADGDYKFACTFPGHGALMNGKITLVD

SEQ ID NO: 13 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKTSMGHNIVIGKTEDMDGIFKDGVGAADTDYVKPDDARVVAHTKLIGGGEESSLTLDPAKLADGEYKFACTFPGHGALMNGKVTLVD

SEQ ID NO: 14 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 15 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEEASLTLDPAKLADGEYKFACTFPGHGALMNGKVTLVD

SEQ ID NO: 16 - protein, 183 aa, Neisseria meningitidis
MKAYLALISAAVIGLAACSQEPAAPAAEATPAAEAPASEAPAAEAAPADAAEAPAAGNCAATVESNDHMQFNTKDIQVSKACKEFTITLKHTGTQPKASMGHNLVIAKAEDMDGVFKDGVGAADTDYVKPDDARVVAHTKLIGGGEESSLTLDPAKLADGEYKFACTFPGHGALMNGKVTLVD

SEQ ID NO: 17 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGEYKFACTFPGHGALMNGKITLVD

SEQ ID NO: 18 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGEYKFACTFPGHGALMNGKVTLVD

SEQ ID NO: 19 - protein, 183 aa, Neisseria meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKTSMGHNLVIAKAEDMDGVFKDGVGAADTDYVKPDDARVVAHTKLIGGGEEASLTLDPAKLADGEYKFACTFPGHGALMNGKVTLVD

SEQ ID NO: 20 - protein, 201 aa, Neisseria gonorrhoeae
MHFDFRKTEYYFIDWRFTMKAYLALISAAVIGLAACSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 21 - protein, 201 aa, Neisseria meningitidis
MHFDFCKTEYYFIDWRFVMKAYLALISAAVIGLAACSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEEASLTLDPAKLADGDYKFACTFPGHGALMNGKVTLVD

SEQ ID NO: 22 - protein, 183 aa, Neisseria gonorrhoeae
MKAYLALISAAVIGLAACSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPGDARVVAHTKLIGGGEESSLTLDPAKLADGDYKFACTFPGHGALMNGKVTLVD

SEQ ID NO: 23 - protein, 183 aa, Neisseria meningitidis
MKAYLALISAAVIGLAACSQEPAAPAAEATPAAEAPASEAPAAEAAPADAAEAPAVGNCAATVESNDNMQFNTKDIQVSKACKEFTITLKHTGTQPKASMGHNLVIAKAEDMDGVFKDGVGAADTDYVKPDDARVVAHTKLIGGGEEASLTLDPAKLADGEYKFACTFPGHGALMNGKVTLVD

SEQ ID NO: 24 - protein, 183 aa, Neisseria meningitidis
MKAYLALISAAVIGLAACSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEEASLTLDPAKLADGDYKFACTFPGHGALMNGKVTLVD

SEQ ID NO: 25 - protein, 183 aa, Neisseria gonorrhoeae
MKAYLALISAAVIGLAACSQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD

SEQ ID NO: 26 - protein, 167 aa, Neisseria
MASQEPAAPAAEATPAGEAPASEAPAAEAAPADAAEAPAAGNCAATVESNDNMQFNTKDIQVSKACKEFTITLKHTGTQPKASMGHNLVIAKAEDMDGVFKDGVGAADTDYVKPDDARVVAHTKLIGGGEESSLTLDPAKLADGDYKFACTFPGHGALMNGKVTLVD